(19) United States
(12) Patent Application Publication
(10) Pub. No.: US 2010/0151544 A1
(43) Pub. Date: Jun. 17, 2010

(54) SYSTEMS AND METHODS FOR SELECTIVE ALCOHOL PRODUCTION

(75) Inventors: Eleftherios T. Papoutsakis, Newark, DE (US); W. Ryan Sillers, Lebanon, NH (US)

Correspondence Address: Casimir Jones, S.C., 2275 DEMING WAY, SUITE 310, MIDDLETON, WI, 53562, US

(73) Assignee: NORTHWESTERN UNIVERSITY, Evanston, IL (US)

(21) Appl. No.: 12/507,645

(22) Filed: Jul. 22, 2009

Related U.S. Application Data
(60) Provisional application No. 61/082,753, filed on Jul. 22, 2008.

(51) Int. Cl. C12P 7/16

(52) U.S. Cl. 435/160

(57) ABSTRACT

The present invention relates to metabolic engineering issues related to flux determinism in core primary-metabolism pathways. In particular, the present invention relates to alcohol (e.g., butanol) production and selectivity, and related methods thereof.

### CROSS-REFERENCE TO RELATED APPLICATIONS

The present application claims the benefit of and priority to pending Provisional Patent Application No. 61/082,753, filed Jul. 22, 2008, the entire disclosure of which is hereby incorporated by reference in its entirety.

### STATEMENT REGARDING FEDERALLY SPONSORED RESEARCH OR DEVELOPMENT

This invention was made with government support under BES-0418157 (CUFS #0830-350-A320) awarded by the NSF. The government has certain rights in the invention.

### FIELD OF THE INVENTION

The present invention relates to metabolic engineering issues related to flux determinism in core primary-metabolism pathways. In particular, the present invention relates to alcohol (e.g., butanol) production and selectivity, and related systems and methods thereof.

### BACKGROUND

Clostridium acetobutylicum, included in the genus Clostridium, is a commercially valuable bacterium. Clostridium acetobutylicum is used to produce acetone, butanol, and ethanol from starch using the ABE process (Acetone Butanol Ethanol process) for industrial purposes such as gunpowder and Cordite (using acetone) production. The A.B.E. process was an industry standard until the late 1940s, when low oil costs drove more-efficient processes based on hydrocarbon cracking and petroleum distillation techniques. C. acetobutylicum also produces acetic acid (vinegar), butyric acid (a substance that smells like vomit), carbon dioxide, and hydrogen. Improved methods for producing butanol from Clostridium acetobutylicum are needed.

### SUMMARY

Metabolic engineering (ME) of Clostridium acetobutylicum has led to increased solvent (e.g., butanol, acetone and ethanol) production and solvent tolerance, thus demonstrating that, for example, further efforts have the potential to create strains of industrial importance. With recently developed ME tools, it is now possible to combine genetic modifications and thus implement more advanced ME strategies.

Experiments conducted during the course of developing embodiments of the present invention demonstrated that antisense RNA (asRNA)-based downregulation of CoA transferase (CoAT, the first enzyme in the acetone-formation pathway) resulted in increased butanol to acetone selectivity, but overall reduced butanol yields and titers. In addition, experiments conducted during the course of developing the present invention demonstrated that the alcohol/aldehyde dehydrogenase (aad) gene (encoding the bifunctional protein AAD responsible for butanol and ethanol production from butyryl-CoA and acetyl-CoA, respectively) was expressed from the phosphotranbutyrylase ptb promoter to enhance butanol formation and selectivity, while CoAT downregulation was used to minimize acetone production. This led to, for example, early production of high alcohol (butanol plus ethanol) titers and overall solvent titers of 30 g/L. Metabolic flux analysis revealed the depletion of butyryl-CoA. In order to increase then the flux towards butyryl-CoA, the impact of thiolase (thl) overexpression was examined. The combined thl overexpression with aad overexpression decreased, as expected, acetate and ethanol production while increasing acetone and butyrate formation.

Accordingly, embodiments of the present invention provide improved methods for alcohol formation and selectivity yields. In some embodiments, the present invention provides, for example, systems and methods to accelerate and enhance alcohol (e.g., butanol) production and selectivity in organisms (e.g., solventogenic clostridia) by, for example, using different combinations of higher aldehyde and alcohol dehydrogenases and/or thiolase expression combined with, for example, CoA transferase downregulation and also by metabolic engineering strategies aimed at, for example, enhancing the flux to and pool of butryl-CoA while minimizing the pool of acetyl-CoA.

In certain embodiments, the present invention provides methods for enhancing butanol production from a bacterial strain. The present invention is not limited to particular methods for enhancing butanol production from a bacterial strain. In some embodiments, the methods involve enhancing butyryl-CoA activity and diminishing acetyl-CoA activity in the bacterial strain for purposes of obtaining increased butanol yield.

The methods are not limited to a particular type of bacterial strain. In some embodiments, the bacterial strain is Clostridium acetobutylicum.

The methods are not limited to a particular manner of enhancing of butyryl-CoA activity. In some embodiments, enhancing of butyryl-CoA activity is accomplished through overexpression of a bifunctional alcohol/aldehyde dehydrogenase gene. The methods are not limited to a particular bifunctional alcohol/aldehyde dehydrogenase gene. Indeed, examples of bifunctional alcohol/aldehyde dehydrogenase genes include the alcohol/aldehyde dehydrogenase (aad) gene.

The methods are not limited to a particular manner of diminishing acetyl-CoA activity. In some embodiments, diminishing acetyl-CoA activity is accomplished through targeting transcripts of enzymes in the acetone formation pathway with antisense RNA. In some embodiments, the antisense RNA is ctfB antisense RNA. In some embodiments, the diminishing of acetyl-CoA activity is accomplished through overexpression of a thiolase gene.

The methods are not limited to a particular manner of regulating overexpession of genes and/or antisense RNA expression. In some embodiments, such regulation is accomplished via a promoter expressed during active cell growth. Examples of promoters expressed during active cell growth include, but are not limited to, a phosphotranbutyrylase (ptb) promoter, a phosphotransacetylase (pta) promoter, and a thiolase (thl) promoter. Any suitable regulatable (e.g., inducible/reproducible) promoter may be used.

In some embodiments, increased butanol and reduced ethanol production in Clostridium acetobutylicum is accomplished through overexpression of the alcohol/aldehyde dehydrogenase gene and the thiolase gene.

In some embodiments, increased butanol and ethanol production in Clostridium acetobutylicum is accomplished through overexpression of the alcohol/aldehyde dehydrogenase gene and through inhibition of acetyl-CoA activity with ctfB antisense RNA.

### BRIEF DESCRIPTION OF THE DRAWINGS

FIG. 1 shows metabolic pathways in C. acetobutyilcum and associated calculated in vivo fluxes. Selected enzymes are shown in bold and associated intracellular fluxes are shown in italics. The metabolic intermediates acetyl-CoA and butyryl-CoA are in ovals to highlight their importance in final product formation. Enzymes are abbreviated as follows: hydrogenase (HYDA); phosphotransacetylase (PTA); acetate kinase (AK); thiolase (THL); β-hydroxybutyryl dehydrogenase (BHBD); crotonase (CRO); butyrylCoA dehydrogenase (BCD); CoA Transferase (COAT); acetoacetate decarboxylase (AADC); butyrate kinase (BK); phophotransbutyrylase (PT B); alcohol/aldehyde dehydrogenase (AAD) Note: AAD is a primary enzyme for butanol and ethanol formation and additional genes exist that code for alcohol forming enzymes (e.g., adhe2, bdhA, bdhB, CAC3292, CAPOO59).

FIG. 2 shows growth and product concentrations of 824(pCASAAD), 824(pAADB1) and 824(pSOS95del) pH 5.0 fermentations. Fermentations were performed in duplicate, while results are shown from one fermentation. Differences in product formation between duplicate fermentations are less than 5%. Lag times were standardized between fermentations by normalizing an A₆₀₀ of 1.0 at hour 10 of the fermentation. 824(pCASAAD) results are shown as open triangles, 824(pAADBI) results are shown as closed squares, and 824(pSOS95del) results are shown as gray circles.

FIG. 3 shows Q RT-PCR analysis of aad expression. Samples were taken from bioreactor experiments shown in FIG. I. A. The ratio of aad expression in 824(pCASAAD) relative to 824(pAADBI) comparing similar timepoints. B The ratio of aad expression in 824(pCASAAD) relative to the first timepoint sampled. C. The ratio of aad expression in 824(pAADBI) relative to the first timepoint sampled.

FIG. 4 shows metabolic flux analysis of 824(pCASAAD), 824(pAADB1) and 824(pSOS95del). 824(pCASAAD) results are shown as open triangles, 824(pAADB1) results are shown as closed squares, and 824(pSOS95del) results are shown as gray circles. Lag times were standardized between fermentations by normalizing an A₆₀₀ of 1.0 at hour 10 of the fermentation.

FIG. 5 shows Metabolic Flux Analysis of 824(pTHLAAD), 824(pPTBAAD) and 824(pSOS95del) 824(pTHLAAD) results are shown as closed circles, 824(pPTBAAD) results are shown as grey squares, and 824(pCASAAD) results are shown as open triangles. Lag times were standardized between fermentations by normalizing an A₆₀₀ of I.0 at hour 10 of the fermentation

FIG. 6 shows growth and product concentrations of 824(pCASAAD), 824(pTHLAAD) and 824(p552) pH 5.0 fermentations, Fermentations were performed in duplicate, while results are shown from one fermentation. Differences in product formation between duplicate fermentations are less than 5%. Lag times were standardized between fermentations by normalizing an A₆₀₀ of 1.0 at hour 10 of the fermentation. 824(pCASAAD) results are shown as open triangles, 824(pTHLAAD) results are shown as closed circles, and 824(pSS2) results are shown as gray diamonds.

### DETAILED DESCRIPTION

Embodiments of the present invention provides systems and methods utilizing Clostridium acetobutylicum and ME techniques for alcohol formation and selectivity yields. In some embodiments, the present invention provides, for example, systems and methods to accelerate and enhance alcohol (e.g., butanol) production and selectivity in organisms (e.g., Clostridium acetobutylicum) by, for example, using different combinations of higher aldehyde and alcohol dehydrogenases and/or thiolase expression combined with, for example, CoA transferase downregulation and also by metabolic engineering strategies aimed at, for example, enhancing the flux to and pool of butryl-CoA while minimizing the pool of acetyl-CoA.

Recent advances in molecular biology and metabolic engineering (ME) techniques involving butyric-acid clostridia offer an opportunity to re-establish acetone, butanol and ethanol (ABE) fermentation as an economically viable process. For example, Clostridium acetobutylicum is a model and prototypical organism for the production of such commodity chemicals (e.g., acetone, butanol, ethanol). In particular, Clostridium acetobutylicum is a model and prototypical organism for the production of butanol, which has, for example, emerged as an important new biofuel. The genome of C. acetobutylicum has been sequenced and annotated (Nolling J, et al., 2001, Journal of Bacteriology 183(6):4823-4838; herein incorporated by reference in its entirety), and methods for genetic deletions (Harris L M, et al., 2002, Journal of Bacteriology 184 (13):3586-3597; Heap J T, et al., 2007, J Microbiol Methods 70(3):452-64; Shao L, et al., 2007, Cell Res 170 1):963-5; each herein incorporated by reference in its entireties) and gene overexpression (Mermelstein L D, et al., 1993, Appl Environ Microbiol 59(4): 107710-81; herein incorporated by reference in its entirety) developed. Furthermore, genome-scale microarray-based transcriptional analyses (Alsaker K V, et al., 2005, J Bacteriol 187(20):7103-18; Alsaker K, et al., 2005, Biotechnology and Bioprocess Engineering 10(5):432-443; Alsaker K V, et al., 2004, Journal of Bacteriology. 186(7):1959-1971; Tomas C A, et al., 2003, Journal of Bacteriology 185(15):4539-4547; Tomas C A, et al., 2003, Journal of Bacteriology 186(7):2006-2018; each herein incorporated by reference in its entirety) have illuminated a complex metabolism, thus allowing the development of precise ME strategies (e.g., through genetic modification strategies).

High butanol selectivity and titers in the ABE fermentation are current obstacles for an economical industrial process. Butanol is a valuable product, and thus minimalized production of all other products is desirable. Ethanol is an additional product that may be desirable as a co-product in the context of biofuel production. ABE batch fermentation is characterized by an acidogenic phase and a solventogenic phase. Initially, the cultures produce the organic acids butyrate and acetate, which lower the culture pH. In the solventogenic phase, the culture produces butanol, acetone, and ethanol. Butyrate and acetate are partially re-assimilated to produce solvents, thus raising the pH of the culture. The trigger responsible for the switch from acid to solvent formation (e.g., known as solventogenesis) has been studied, but the exact mechanism for this change remains unknown. The external pH is known to affect solventogenesis and product formation (Husemann M H W, et al., 1988, Biotechnology and Bioengineering 32(7): 843-852; herein incorporated by reference in its entirety). Recent evidence correlates increases of butyryl-phosphate (BuP) concentration with the onset of solvent formation and suggests that BuP performs a role in the regulation of solvent initiation (Zhao Y S, et al., 2005, Appl. Environ Microb. 71(1):530-537; herein incorporated by reference in its entirety).

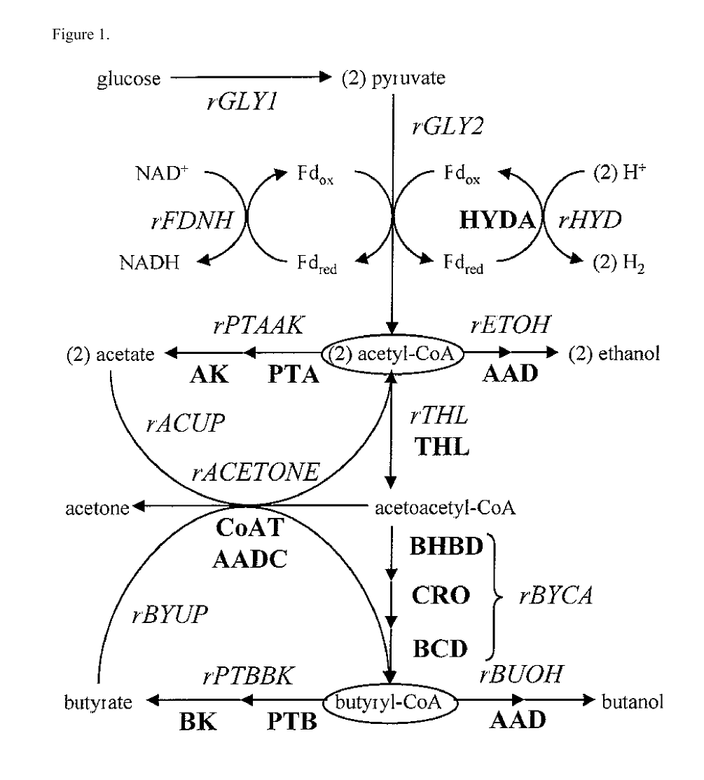

In wild-type C. acetobutylicum fermentations, final acetone concentrations are typically one-half the final levels of butanol. Initial efforts to increase the selectivity of butanol to acetone used antisense RNA (asRNA) technology targeting the transcripts of enzymes in the acetone formation pathway (see FIG. 1). The ctfB asRNA successfully reduced acetone production when designed to downregulate a subunit of the first enzyme in the acetone formation pathway, CoA transferase (CoAT) (Tummala S B, et al., 2003, Journal of Bacteriology 185(6): 1923-1934; herein incorporated by reference in its entirety). However, butanol titers were also significantly reduced in the ctfB asRNA strain. The ctfB gene is part of a tricistronic operon (aad-ctfA-ctfB) also containing the aad gene, whose product, the bifunctional AAD (aldehyde-alcohol dehydrogenase) protein, catalyzes the two-step conversion of butyryl-CoA to butanol or of acetyl-CoA to ethanol (Nair R V, et al., 1994, Journal of Bacteriology 176(3):871-885; herein incorporated by reference in its entirety). Because the ctfB and aad genes reside on the same mRNA transcript, the ctfB asRNA resulted in a downregulation of both the ctfB and aad genes thus resulting in lower butanol production (Tummala S B, et al., 2003, Journal of Bacteriology 185(6): 1923-1934; herein incorporated by reference in its entirety). Follow-up studies were able to restore wild-type butanol titer levels while maintaining low acetone production by combining, in strain 824(pAADB1) the ctfB asRNA with the overexpression of the aad gene alone off plasmid pAADB1 using its own autologous promoter (Tummala S B, et al., 2003, Journal of Bacteriology 185(12):3644-3653; herein incorporated by reference in its entirety). Significantly, this strain produced ca. 200 mM ethanol, a very high result for C. acetobutylicum. The high ethanol production was due to the dual functionality of the AAD enzyme, which catalyzes both the formation of ethanol and butanol. In the wild-type strain, butanol is produced nearly six-fold higher than ethanol. AAD has, for example, a much higher affinity for butyryl-CoA than for acetylCoA (FIG. 1). The high ethanol production by strain 824(pAADBI) suggests that, for example, the ratio of acetyl-CoA to butyryl-CoA is much higher in this strain than in the wild-type strain.

Experiments conducted during the course of development of embodiments for the present invention demonstrated ME strategies resulting in enhanced butanol formation and selectivity and, significantly, accelerated butanol production. In particular, experiments demonstrated that regulation of fluxes around the two critical nodes of butyryl-CoA and acetyl-CoA (FIG. 1) resulted in increased butanol production and diminished acetone production. In particular, aad (AAD) overexpression by changing the temporal expression of this gene using the ptb promoter (of the ptb-buk operon) coding the two enzymes responsible for butyrate production from butyryl-CoA (see FIG. 1) which is expressed early in the acidogenic growth phase (Tummala S B, et al., 1999, Applied and Environmental Microbiology 65(9):3793-3799; herein incorporated by reference in its entirety) when the aad natural expression is normally absent (Nair R V, et al., 1994, Journal of Bacteriology 176(3):871-885; herein incorporated by reference in its entirety) was demonstrated. Early expression of aad sought to direct more of the carbon flux towards butanol production while limiting the formation of butyrate by competing early for butyryl-CoA. In addition, reduction of ethanol and acetate production by altering the fluxes around the acetyl-CoA node focusing on the overexpression of the thiolase gene (FIG. 1) was also demonstrated.

Accordingly, in some embodiments, the present invention provides methods for enhancing alcohol formation (e.g., ethanol and butanol) from a bacteria strain (e.g., Clostridium acetobutylicum). The present invention is not limited to particular methods for enhancing and acceleration alcohol production from a bacterial strain (e.g., a solventogenic clostridium strain). In some embodiments, the methods comprise enhancing butyryl-CoA activity and diminishing acetyl-CoA activity.

The present invention is not limited to a particular bacteria strain. In some embodiments, the bacteria strain is E. coli. In some embodiments, the bacterial strain is a solventogenic clostridium strain. In some embodiments, the solventogenic clostridium strain is Clostridium acetobutylicum.

The present invention is not limited to a particular method for enhancing butyryl-CoA activity. In some embodiments, enhancement of butyryl-CoA activity is achieved through overexpression of a bifunctional alcohol/aldehyde dehydrogenase gene responsible for butanol and ethanol production from butyryl-CoA and acetyl-CoA. The present invention is not limited to a particular bifunctional alcohol/aldehyde dehydrogenase gene responsible for butanol and ethanol production from butyryl-CoA and acetyl-CoA. Examples include, but are not limited to, CAP0162 from the C. acetobutylicum genome, CAP0035 C. acetobutylicum genome, CAC3298 C. acetobutylicum genome, CAC3299 C. acetobutylicum genome, CAC3292 C. acetobutylicum genome, and CAP0059 C. acetobutylicum genome. In some embodiments, the gene responsible for butanol and ethanol production from butyryl-CoA and acetyl-CoA is alcohol/aldehyde dehydrogenase (aad) gene.

The present invention is not limited to a particular method for diminishing (e.g., inhibiting, reducing) acetyl-CoA activity. In some embodiments, diminishing acetyl-CoA activity is accomplished through targeting the transcripts of enzymes in the acetone formation pathway (see, e.g., FIG. 1) with antisense RNA (asRNA). In some embodiments, ctfB asRNA is used to block acetyl-CoA activity. In some embodiments, diminishing acetyl-CoA activity is accomplished through overexpression of the thiolase (thl) gene.

In some embodiments, overexpression of a bifunctional alcohol/aldehyde dehydrogenase gene responsible for butanol and ethanol production from butyryl-CoA and acetyl-CoA (e.g., the aad gene) is regulated via a promoter expressed during active cell growth. In some embodiments, asRNA targeting enzymes in the acetone formation pathway is regulated via a promoter expressed during active cell growth. In some embodiments, a promoter expressed during active cell growth is a phosphotranbutyrylase (ptb) promoter (e.g., of the ptb-buk operon coding two enzymes responsible for butyrate production from butyryl-CoA; see, e.g., FIG. 1). In some embodiments, a promoter expressed during active cell growth is a phosphotransacetylase (pta) promoter. In some embodiments, a promoter expressed during active cell growth is a thiolase (thl) promoter. As such, in some embodiments, enhancement of butyryl-CoA activity is achieved through overexpression of a bifunctional alcohol/aldehyde dehydrogenase gene responsible for butanol and ethanol production from butyryl-CoA and acetyl-CoA (e.g., aad) driven via a promoter expressed during active cell growth (e.g., ptb, pta, thl). In some embodiments, asRNA targeting enzymes in the acetone formation pathway is driven via a promoter expressed during active cell growth (e.g., ptb, pta, thl). In some embodiments, overexpression of thiolase gene (thl) for purposes of diminishing acetyl-CoA activity is driven via a promoter expressed during active cell growth (e.g., ptb, pta, thl). In some embodiments, targeting enzymes in the acetone formation pathway is driven via a promoter expressed during active cell growth (e.g., ptb, pta, thl). In some embodiments, 1) overexpression of a bifunctional alcohol/aldehyde dehydrogenase gene responsible for butanol and ethanol production from butyryl-CoA and acetyl-CoA (e.g., aad), 2) overexpression of thiolase gene (thl) for purposes of diminishing acetyl-CoA activity, and/or 3) asRNA targeting enzymes in the acetone formation pathway are driven via a promoter expressed during active cell growth (e.g., ptb, pta, thl).

In some embodiments, the methods for obtaining enhanced alcohol formation is further accomplished through overexpressing one or more genes coding for proteins responsible for butyryl-CoA formation from acetoacetyl-CoA. The methods are not limited to particular genes coding for proteins responsible for butyryl-CoA formation from acetoacetyl-CoA. In some embodiments, genes coding for proteins responsible for butyryl-CoA formation from acetoacetyl-CoA include, but are not limited to, hbd, etfA, etfB, bcd, and cro. In some embodiments, overexpression of one or more genes coding for proteins responsible for butyryl-CoA formation from acetoacetyl-CoA is regulated via one or more promoters expressed during active cell growth (e.g., ptb, pta, thl).

In some embodiments, the methods for obtaining enhanced alcohol production further involve inhibition of ethanol production so as to obtain higher butanol yield. The methods are not limited to a particular manner of inhibiting ethanol production so as to obtain higher butanol yield. In some embodiments, inhibition of ethanol production so as to obtain higher butanol yield is accomplished through downregulation and/or knockout of pyruvate decarboxylase (PDC). In some embodiments, inhibition of ethanol production so as to obtain higher butanol yield is accomplished through overexpression of thiolase (thl) gene. Indeed, in some embodiments, the methods further comprise overexpression of any suitable thiolase gene/protein to enhance the flux from acetyl-CoA to acetoacetyl-CoA and thus minimize the acetylCoA pool. In some embodiments, the methods employ suitable thiolase genes which have been protein engineered by standard methods to generate a thiolase gene with an extremely small Km value for acetyl-CoA in order to drive the acetyl-CoA to acetoacetyl-CoA faster and lower acetyl CoA intracellular pools and thus further minimize the acetyl-CoA pool and thus minimize ethanol production.

### EXAMPLES

### Bacterial Stains and Plasmids

The list of bacterial strains and plasmids are in Table I.

TABLE I
Bacterial stains and plasmids used in this study.

| Strain or Plasmid | Relevant Characteristics^{a} | Source or Reference^{b} |
|---|---|---|
| Bacterial Strains | | |
| C. acetobutylicum | | |
| ATCC 824 | | ATCC |
| M5 | | (Clark et al. 1989) |
| E. coli | | |
| Top10 | | Invitrogen |
| ER2275 | | New England Biolabs |
| Plasmids | | |
| pAN1 | Cm^{r}, Φ3T I gene, p15A | (Mermelstein and |
| | origin | Papoutsakis 1993) |
| pSOS94^{c} | acetone operon (ptb | Soucaille and |
| | promoter) | Papoutsakis, |
| | | unpublished |
| p94AAD3^{c} | aad, (ptb promoter) | This study |
| pCTFB1AS^{c} | ctfB asRNA (thl promoter) | (Tummala et al 2003b) |
| pCASAAD^{c} | aad, (ptb promoter), ctfB | This study |
| | asRNA (thl promoter) | |
| pAADB1^{c} | aad, (aad promoter), ctfB | (Tummala et al. 2003a) |
| | asRNA (thl promoter) | |
| pTHL^{c} | thl | This study |
| pTHLAAD^{c} | thl, aad, (ptb promoter) | This study |
| pPTBAAD^{c} | aad, (ptb promoter) | This study |
| pCAS^{c} | ctfB asRNA (adc promoter) | This study |
| pSOS95del^{c} | thl promoter | (Tummala et al 2003a) |
| pSS2^{c} | aad, (ptb promoter), ctfB | This study |
| | asRNA (adc promoter), thl | |

^{a}Cm^{r}, chloramphenicol resistance gene; ptb, phosphotransbutyrylase gene; aad, alcohol/aldehyde dehydrogenase gene; ctfB, CoA transferase subunit B gene; thl, thiolase gene; adc, acetoacetate decarboxylase gene
^{b}ATCC, American Tissue Culture Collection, Rockville, MD
^{c}contans the following: ampicillin resistance gene; macrolide, lincosimide, and streptogramin B resistance gene: repI, pIM13 Gram-positive origin of replication; ColE1 origin of replication

### Culture Conditions

E. coli strains were grown aerobically at 37° C. and 200 rpm in liquid LB media or solid LB with agar (1.5%) media supplemented with the appropriate antibiotics (ampicillin at 50 μg/mL or chloramphenicol at 35 μg/mL). Frozen stocks were made from 1 mL overnight culture resuspended in LB containing 15% glycerol and stored at −85° C. C. acetobutylicum strains were grown anaerobically at 37° C. in an anaerobic chamber (Thermo Forma, Waltham, Mass.). Cultures were grown in liquid CGM (containing 0.75 g KH₂PO₄, 0.982 g K₂HPO₄, 1.0 g NaCl, 0.01 g MnSO₄, 0.004 g PABA, 0.348 g MgSO₄, 0.01 g FeSO₄, 2.0 g asparagine, 5.0 g yeast extract, 2.0 g (NH₄)₂S0₄, and 80 g glucose, all per liter) media or solid 2×YTG pH 5.8 (containing 16 g Bacto tryptone, 10 g yeast extract, 4 g NaCl, and 5 g glucose, all per liter) plus agar (1.5%) supplemented with antibiotics as necessary (erythromycin at 100 μg/mL in liquid media and 40 μg/mL in solid media, clarithromycin at 75 μg/mL). Cultures were heat shocked at 70-80° C. for 10 minutes prior to enhance solvent production and prevent strain degeneration (Cornillot E, et al., 1997, J. Bacteriol. 179(17):5442-5447; herein incorporated by reference in its entirety). Frozen stocks were made from 10 mL of A₆₀₀=1.0 culture resuspended in 1 mL CGM containing 15% glycerol and stored at −85° C.

### Plasmid and Strain Construction

The aad gene (CAP0162) responsible for butanol formation was PCR amplified from C. acetobutylicum genomic DNA using primers aad_fwd and aad_rev to exclude the natural promoter. All primers used in plasmid construction are listed in Table II. The pSOS94 vector was digested with BamHI and EheI and blunt ended to remove the acetone formation genes while leaving the ptb promoter region and the adc terminator. The aad PCR product and the linearized pSOS94 vector were ligated to create p94AAD3. Both pCTFB1AS, containing the ctfB asRNA, and p94AAD3 were digested with SalI to linearize pCTFB1AS and isolate the aad gene with the ptb promoter and adc terminator from p94AAD3. These fragments were ligated together to generate pCASAAD.

TABLE II
List of primers and oligonucleotides.

| | | | SEQ |
|---|---|---|---|
| Primer | | | ID |
| Name | Sequence (5′-3′) | Description | NO |
| aad_fwd | TTAGAAAGAAGTGTATATTTAT | aad forward | 1 |
| | | primer | |
| aad_rev | AAACGACGGCCAGTGAAT | aad reverse | 2 |
| | | primer | |
| thl_fwd | CCATATGTCGACGGAAAGGCTTCA | thl forward | 3 |
| | | primer | |
| thl_rev | ACGCCTAGTACTGAATTCGCCTCA | thl reverse | 4 |
| | | primer | |
| p_adc_top | TCGACTAAAAATTTACTTAAAAAA | adc | 5 |
| | ACATATGTGTTATAATGAAATATA | promoter | |
| | AATAAATAGGACTAGAGGCGATTT | top oligo- | |
| | ATAATGTGAAGATAAAGTATGTTA | nucleotide | |
| | G | | |
| p_adc_bot | AATTCTAACATACTTTATCTTCAC | adc promoter | 6 |
| | ATTATAAATCGCCTCTAGTCCTAT | bottom | |
| | TTATTTATATTTACATTATAACAC | oligonucleo- | |
| | ATATTGTTTTTTTAAGTAAATTTT | tide | |
| | TAG | | |
| ctfBas_top | AATTCTTAATTCTCTTGCAACTCT | ctfB asRNA | 7 |
| | TTTGGCTATTATTTCTTTCGCTAG | top oligo- | |
| | GTTTTTATCATTAATCATTTTATG | nucleotide | |
| | CAGGCTCCTTAAAAGTAATTACAT | | |
| | TACA | | |
| ctfBas_bot | TATGATATGTAATTACTTTTAAGG | cftB asRNA | 8 |
| | AGCCTGCATAAAATGATTAATGAT | bottom | |
| | AAAAACCTAGCGAAAGAAATAATA | oligo- | |
| | GCCAAAAGAGTTGCAAGAGAATTA | nucleotide | |
| | AG | | |
| cas_fwd | TCGACTAAAAATTTACTTAAAAAA | cftB asRNA | 9 |
| | AC | forward | |
| | | primer | |
| cas_rev | TATGATATGTAATTACTTTTAAGG | cftB asRNA | 10 |
| | | reverse | |
| | | primer | |
| aad_rt_fwd | AGAAAATGGCTCACGCTTCA | aad RT-PCR | 11 |
| | | forward | |
| | | primer | |
| aad_rt_rev | GCAATGCCAACTAGGAATATTGTG | aad RT-PCR | 12 |
| | | reverse | |
| | | primer | |
| pul_rt_fwd | TTCTCCACTGTGGCGTAGAGTT | thl RT-PCR | 13 |
| | | forward | |
| | | primer | |
| pul_rt_rev | TCTCTAAGATCCCAATCTATCCAA | thl RT-PCR | 14 |
| | TTT | reverse | |
| | | primer | |

The thiolase (thl) gene including the endogenous promoter and terminator regions was amplified from C. acetobutyiicum genomic DNA using primers thl_fwd and thl_rev. Following purification, the PCR product was digested with SalI and EcoRI as was the shuttle vector pIMP1. The digested PCR product was ligated into the pIMP1 shuttle vector to form the plasmid pTHL. The aad gene cassette from p94AAD3 was isolated using a SalI digestion and purified. Plasmid pTHL was SalI digested and ligated with the purified aad gene cassette to generate plasmid pTHLAAD

A revised ctfB asRNA cassette was generated by first inserting a 100 by oligonucleotide into the pIMP1 shuttle vector following digestion with SalI and EcoRI. This oligonucleotide includes the sequence for the adc promoter element with compatible nucleotide overhangs for ligation. The complimentary oligonucleotides p_adc_top and p_adc_bot were first annealed together before ligating into the pIMP1 vector, creating pPADC, which was then digested with EcoRI and NdeI. A second set of complementary oligonucleotides, ctfBas_top and ctfBas_bot, were annealed and ligated to the digested pPADC to form pCAS. The new ctfB asRNA cassette was PCR amplified from this plasmid using primers cas_fwd and cas_rev and ligated into the pTHLAAD plasmid to generate plasmid pSS2.

All plasmids were transformed into Top 10 chemically competent E. coli (Invitrogen, Carlsbad, Calif.). Plasmids were confirmed using sequencing reactions. The plasmids were methylated using E. Coli ER2275 (pAN 1) cells to avoid the natural restriction system of C. acetobutylicum (Mermelstein L D, et al., 1993, Appl Environ Microbiol 59(4): 107710-81; herein incorporated by reference in its entirety). Once methylated, the plasmids were transformed by electroporating C. acetobutylicum wildtype or mutant M5 strains as described (Mermelstein L D, et al., 1992, Biotechnology (NY) 10(2):190-5; herein incorporated by reference in its entirety).

### Bioreactor Experiments

Fermentations were carried out using a BioFlo 110 or BioFlo II (New Brunswick Scientific Co., Edison, N.J.) bioreactor with 4.0 L working volumes. Fermentations used a 10% v/v inoculum of a pre-culture with A₆₀₀ equal to 0.2. CGM media were supplemented with 0.10% (v/v) antifoam and 75 μg/mL clarithromycin. Fermentations were maintained at constant pH using 6 M NH₄OH. Anaerobic conditions were maintained through nitrogen sparging. Temperature was maintained at 37° C. and agitation was set at 200 rpm. Glucose was restored to the initial concentration (440 mM) in fermentations if glucose levels fell below 200 mM.

### Analytical Techniques

Cell density was measured at A₆₀₀ using a Biomate3 spectrophotometer (Thermo Spectronic, Waltham, Mass.). Samples were diluted as necessary to keep absorbance below 0.40. Supernatant concentrations of glucose, acetone, acetate, acetoin, butyrate, butanol, and ethanol were determined using a high-pressure liquid chromatography system (HPLC) (Waters Corp. Milford, Mass.) (Buday Z, et al., 1990, Enzyme and Microbial Technology 12(1):24-27; herein incorporated by reference in its entirety). Mobile phase of 0.15 mM H₂S0₄ at 0.50 mL/min was used with an Aminex HPLC Organic Acid Analysis Column (Biorad, Hercules, Calif.) The column was cooled to 15° C. and samples were run for 55 minutes.

### RNA Sampling and Isolation

Cell pellets from 3 to 10 mL of culture were incubated at 37° C. for 4 minutes in 200 μL of SET buffer (25% sucrose, 50 mM EDTA pH 80, 50 mM Tris-HCl pH 8.0) with 20 mg/mL lysozyme. 1 mL trizol was added to each sample and stored at −85° C. until purification. 0.5 mL Trizol and 0.2 mL chloroform was added to 0.5 mL RNA sample and centrifuged at 12,000 rpm for 15 minutes. The aqueous phase was collected and added to an equal volume of isopropanol and RNA was precipitated at 12,000 rpm for 10 minutes. 1 ml, ethanol was added to wash the pellet and centrifuged at 9,500 rpm for 4 minutes. Samples were dried and resuspended in 20-100 μL of RNase free water and stored at −85° C.

### Quantitative (Q)-RT-PCR

Reverse transcription of RNA was carried out using random hexamer primers with 500 μM dNTPs, 2.0 μg RNA, 2 μL RNase inhibitor, 2.5 μL reverse transcriptase, and 2.5 μM random hexamers in a total volume of 100 μL (Applied Biosystems). The reaction was incubated at 25° C. for 10 minutes, 48° C. for 30 minutes, followed by inactivation of the enzymes by a five-minute incubation at 95° C. The SYBR green master mix kit (Applied Biosystems) was used for RT-PCR Each PCR contained 1 μL cDNA and 1 μM gene specific pnmers (Table 2) in a total volume of 25 μL. Samples were performed in tnplicate on a BioRad iCycler with the following parameters: 10 minutes at 95° C., forty cycles of 15 sec at 95° C. and 1 minute at 60° C. All genes were normalized to the pullulanase gene (Tomas C A, et al., 2003, Appl. Environ. Microb. 69(8):4951-4965; herein incorporated by reference in its entirety).

### Metabolic Flux Analysis

Metabolic Flux analysis calculations were per for med using a program developed by Desai et al (Desai R P, et al., 1999, Journal of Biotechnology 71:191-205; herein incorporated by reference in its entirety). Product concentrations from bioreactor experiments were used to generate metabolic fluxes. Error associated with the calculated fluxes is typically less than 10 percent.

### Early and Elevated Expression of aad Using the ptb Promoter

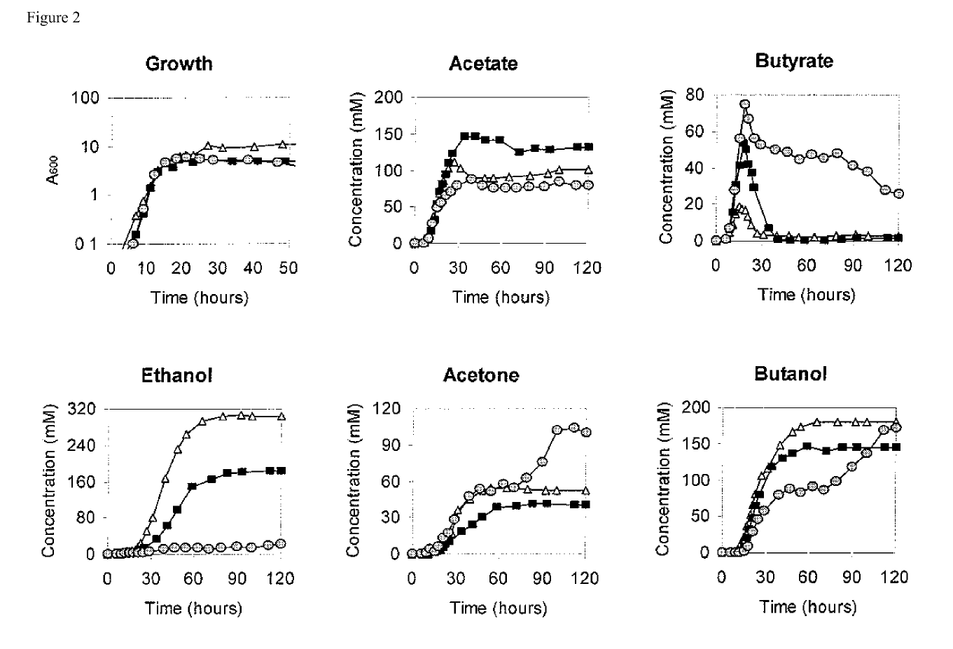

ME strategies to enhance butanol formation and selectivity and accelerate butanol production were explored. The regulation of fluxes around the two critical nodes of butyryl-CoA and acetyl-CoA was explored (FIG. 1). First, butanol and ethanol production was enhanced and accelerated by increasing the expression of the enzyme responsible for butanol and ethanol formation, alcohol/aldehyde dehydrogenase (AAD) (FIG. 1). This was accomplished by changing the temporal expression of this gene using the ptb promoter, p_{ptb} (of the ptb-buk operon, coding the two enzymes responsible for butyrate production from butyryl-CoA), which is active early in the acidogenic growth phase (Tummala S B, et al., 1999, Applied and Environmental Microbiology 65(9):3793-3799; herein incorporated by reference in its entirety) when the aad natural expression is normally absent (Nair R V, et al., 1994, Journal of Bacteriology 176(3):871-885; herein incorporated by reference in its entirety) This early expression of aad sought to direct mote of the carbon flux towards butanol production while limiting the formation of butyrate by competing early for butyryl-CoA. The combination of the early plasmid-expressed aad with the later chromosomally-expressed aad from its natural promoter should allow for the sustained butanol production throughout both the acidogenic and solventogenic growth phases. Plasmid p94AAD3 was created to express aad from the p_{ptb} p94AAD3 was first transformed into the degenerate strain M5 (which has lost the pSOL1 megaplasmid and thus the ability to express the sol operon and form butanol or acetone (Cornillot E, et al., 1997, J. Bacteriol. 179(17):5442-5447; herein incorporated by reference in its entirety) to confirm the proper expression of aad and the production of a functional protein. Production of butanol in M5(p94AAD3) was observed confirming the proper expression and translation of the aad gene from p_{ptb}. Then, aad expressed from the p_{ptb} was isolated from p94AAD3 and combined into a plasmid containing the ctfB asRNA, creating plasmid pCASAAD. Following the transformation of pCASAAD into the wild-type strain, controlled pH 5.0 fermentations were performed in duplicate to fully characterize the 824(pCASAAD) compared to the strain containing the ctfB asRNA and aad overexpression from its endogenous promoter, 824(pAADB1), and the plasmid control strain 824(pSOS95del) (FIG. 2).

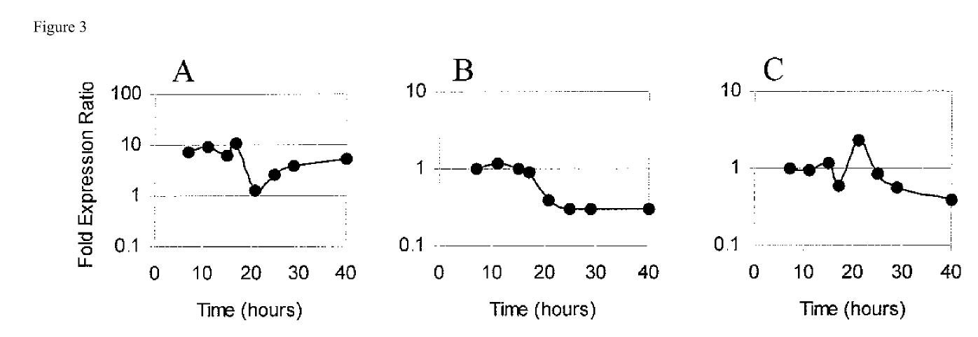

RNA samples were collected during the fermentations and analyzed for the level of aad expression using Q-RT PCR. Comparing the aad expression between the strains, there exists a nearly ten-fold higher expression of aad in 824(pCASAAD) than in 824(pAADB1) during the first four timepoints (FIG. 3). These timepoints correspond to the exponential growth phase and the early transitional phase when the p_{ptb} is expected to have the highest activity. During the later timepoints aad expression continues to be higher in 824(pCASAAD), but at lower levels than initially observed. The expression of aad within each strain was also examined. In 824(pCASAAD), aad expression is highest during the first four timepoints after which the expression level decreases. This pattern is the opposite of the wild-type strain where aad expression is absent early, but is later induced in stationary phase (Alsaker K, et al., 2005, Biotechnology and Bioprocess Engineering 10(5):432-443; herein incorporated by reference in its entirety). This shows that, for example, the p_{ptb} was successful in enhancing the early expression of aad. The pattern of aad expression in 824(pAADB1) is more complex. There exists a distinct peak in expression of aad that corresponds to the entry into stationary phase, when aad is induced in the wild-type strain. After this point the aad expression begins to decrease.

ptb-Promoter-Driven aad Expression Leads to Higher Cell Densities and Increased, Earlier Butanol Formation

Although the growth rate was similar between all strains, 824(pCASAAD) reached higher cell densities (Table III) than either 824(pAADB1) or 824(pSOS95del).

TABLE III
Product formation in pH 5.0 fermentation experiments

| | Fermentation characteristics^{a} | | | | | | | |
|---|---|---|---|---|---|---|---|---|
| Strains | Max A₆₀₀ | Butanol | Ethanol | Acetone | Acetateₚₑₐₖ | Acetate_{final} | Butyrateₚₑₐₖ | Butyrate_{final} |
| 824(pSOS95del) | 5.79 | 176 | 19 | 109 | 80 | 77 | 73 | 37 |
| 824(pAADB1) | 5.60 | 146 | 184 | 42 | 147 | 129 | 53 | 1 |
| 824(pCASAAD) | 11.80 | 178 | 300 | 61 | 105 | 85 | 20 | 2 |
| 824(pTHL) | 10.67 | 54 | 6 | 34 | 68 | 68 | 76 | 71 |
| 824(pTHLAAD) | 9.75 | 153 | 28 | 98 | 68 | 67 | 39 | 17 |
| 824(pPTBAAD) | 11.90 | 160 | 76 | 59 | 124 | 124 | 62 | 2 |
| 824(pSS2) | 10.70 | 137 | 288 | 29 | 120 | 106 | 16 | 2 |
| 824(pCAS)^{b} | 3.59 | 53 | 11 | 29 | 38 | 38 | 45 | 34 |
| 824(pSOS95del)^{b} | 4.35 | 152 | 21 | 91 | 22 | 12 | 33 | 22 |

^{a}All results shown are average mM concentration from duplicate experiments
^{b}Results are from static flask experiments without pH control

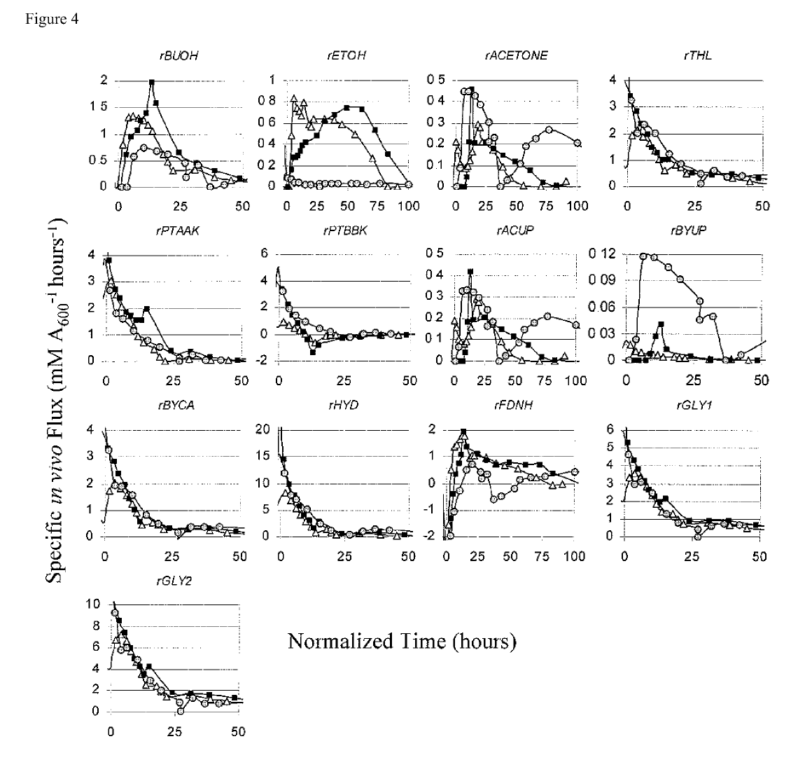

These higher cell densities were attributed to, for example, the lower butyrate concentrations observed in the 824(pCASAAD) strain (FIG. 2): butyrate was completely re-assimilated by both strains with the ctfB asRNA, but peak butyrate levels were reduced by two-thirds in 824(pCASAAD) compared to 824(pAADB1). Furthermore, both the peak and final acetate levels were reduced in 824(pCASAAD) compared to 824(pAADBI): final acetate concentrations were 129 mM in 824(pAADB1), 85 mM in 824(pCASAAD) and 77 mM in the plasmid control strain 824(pSOS95del). The solvent formation profiles also show significant differences between strains. In the control 824(pSOS95del) strain, acetone and butanol are the primary solvents produced, 109 mM and 176 mM respectively, while ethanol formation is relatively minor, at about 20 mM. The acetone production of 824(pCASAAD) is slightly higher than in 824(pAADB1), but 824(pSOS95del) produces twice the acetone of 824(pCASAAD). Butanol production was higher in 824(pCASAAD) (178 mM) and 824(pSOS95del) than in 824(pAADB1) (146 mM). Significantly, butanol was produced earlier and reached its final levels in half the time in 824(pCASAAD) (about 60 hrs.) than in 824(pSOS9Sdel) (about 120 hrs.) (FIG. 2). Earlier butanol formation is better demonstrated in the plots showing the specific intracellular fluxes (FIG. 4). Ethanol production was dramatically higher in 824(pCASAAD) and 824(pAADB1) than in 824(pSOS95del). 824(pCASAAD) produced 305 mM ethanol, 15 times higher than the control strain, while 824(pAADB1) produced 184 mM. This is the highest ethanol production reported by any solventogenic clostridium. These data show that, for example, butyryl-CoA and acetyl-CoA are important determinants of solvent yields and selectivities, and this is further illuminated by metabolic flux analysis which is presented next.
Metabolic Flux Analysis of the Three Strains Supports the Limiting Role of Butyryl-CoA and Acetyl-CoA for Butanol Vs. Ethanol Production, Respectively

Using a previously developed model (Desai R P, et al., 1999, Appl Environ Microbiol 65(3):936-45; herein incorporated by reference in its entirety), the fluxes of 824(pAADB1) and 824(pCASAAD) were calculated and normalized both for differences in lag times of growth and cell density. First, the core carbon fluxes GLY 1, GLY 2, thiolase and BYCA, and the H₂ formation flux were largely similar among the three strains (except for the first 3-4 normalized hours in strain 824(pCASAAD), which were lower, likely due to the metabolic burden of the early AAD overexpression), and thus unaffected by the genetic modifications, which is theoretically expected and a desirable finding. The butanol and ethanol formation fluxes show significantly higher values early in 824(pCASAAD) than in 824(pAADB1) or the plasmid control. This is consistent with the observation that the FDNH fluxes (NADH₂ production from reduced ferredoxin coupled to the GLY 2 flux (FIG. 1)) show higher values earlier in the order (high to low) of 824(pCASAAD), 824(pAADB1) and 824(pSOSdel). In strain 824(pCASAAD) the butanol formation flux dropped to less than 25% its maximum at 21 hours, while the ethanol formation flux is maintained at over 50% its maximal value for nearly 60 hours. Although the peak formation values occur later in 824(pAADB1), the same trends of butanol and ethanol production are evident. In 824(pAADB1), the ethanol formation flux only reaches its maximum value after the butanol formation flux sharply decreases at 25 hours. Butanol formation precedes ethanol formation in all strains, but ethanol formation is sustained for longer time periods than butanol formation, and especially so after 25 hours when the flux (BYCA) to butyryl-CoA as well as the acetate and butyrate fluxes have largely been reduced to zero, while the GLY 2 flux (FIG. 1) remains still at reasonable levels (about 1 mM A₆₀₀⁻¹ h⁻¹; FIG. 3). This suggests that, for example, butyryl CoA availability limits butanol formation, while the substantial flux to acetyl-CoA combined with high levels of AAD expression feeds and sustains the flux to ethanol at high levels thus leading to the very high ethanol titers. The nearly zero flux of acetate formation after 25 hours and the sustained high ethanol fluxes late past 50 to 60 hours suggest that the high AAD activity combined with the likely lower activities of the acetate formation enzymes are responsible for channeling most of the carbon to ethanol by utilizing all the available reducing power and thus the zero H₂ formation flux at that time period.

The butyrate formation flux is particularly low in 824(pCASAAD), thus demonstrating, for example, that the strategy for channeling butyryl-CoA from butyrate to butanol formation by the early and strong aad overexpression has worked as anticipated. Due to the low butyrate formation, butyrate uptake is much lower in 824(pCASAAD). Acetate formation is also sustained better and longer in 824(pAADB1) than in 824(pCASAAD) and the plasmid-control strain, and this is consistent with the deduced longer sustained acetyl-CoA pool that sustains much longer a high ethanol flux. Comparing the acid uptake fluxes with the acetone formation flux, it is evident that acetone is produced from the uptake of acetate, as the acetate uptake flux is 10-fold higher than the butyrate uptake flux in both 824(pAADB1) and 824(pCASAAD). Acetone formation is also sustained longer in 824(pAADB1) than in 824(pCASAAD), but both strains show the anticipated lower acetone fluxes compared to the plasmid-control strain as a result of the asRNA downregulation of the acetone-formation enzyme CoAT (FIGS. 1 and 3).

### Role of Thiolase Promoter and Thiolase Expression on the Acetyl-CoA to Butyryl-CoA Flux, and its Impact on Product Formation.

The data discussed above (FIGS. 3 and 4) suggest, for example, there exists a bottleneck for the formation of butyryl-CoA from acetyl-CoA. Four enzymes catalyze the conversion of butyryl-CoA from acetyl-CoA (FIG. 1) These are organized into two operons on the chromosome. The first enzyme in the pathway, thiolase (coded by the monocistronic thl), converts acetyl-CoA to acetoacetyl-CoA. The other three enzymes, 3-hydroxybutryl-coenzyme A dehydrogenase (BHBD), crotonase (CR0), and butyryl-CoA dehydrogenase (BCD), convert acetoacetyl-CoA to butyryl-CoA and are co-transcribed as a single, large operon (Boynton Z L, et al., 1996, J. Bacteriol. 178(11):3015-3024; herein incorporated by reference in its entirety). thl is expressed at high levels. Its constitutive-like expression (Tummala S B, et al., 1999, Applied and Environmental Microbiology 65(9):3793-3799; herein incorporated by reference in its entirety) makes it an ideal promoter (pₜₕₗ) for high expression studies in clostridia, and was thus used to drive the expression of the ctfB asRNA in these studies. It is possible that the use of pₜₕₗ for the ctfB asRNA could have lowered the expression of the endogenous thl gene, thereby lowering THL activity and creating the acetyl-CoA buildup. In order to test this hypothesis, the ctfB asRNA was expressed from another promoter, namely the promoter of the acetoacetate decarboxylase (FIG. 1) gene adc (p_{adc}), which is also highly expressed but a little later than pₜₕₗ (Tummala S B, et al., 1999, Applied and Environmental Microbiology 65(9):3793-3799; herein incorporated by reference in its entirety), and since it is used in the formation of acetone, any promoter titration effects would not negatively impact butanol formation. A 100-basepair oligonucleotide was designed to include the integral portions of the adc promoter (Gerischer U, et al., 1990, J Bacteriol 172(12):6907-6918; herein incorporated by reference in its entirety). Following its ligation into the pIMP 1 shuttle vector, another oligonucleotide was designed to include the Shine-Delgarno sequence of the ctfB gene and about 50 basepairs of the downstream coding sequence. Following the ctfB sequence was the glnA hairpin terminator (Desai R P, et al., 1999, Appl Environ Microbiol 65(3):936-45; herein incorporated by reference in its entirety). This new ctfB asRNA was ligated downstream of p_{adc} to create the plasmid pCAS (Table I). This plasmid was transformed into the wild-type strain to confirm the functionality of the new ctfB asRNA. This new strain (824(pCAS)) was characterized in static fermentations and compared to the original ctfB asRNA. Acetone formation was much lower in 824(pCAS) than in the 824(pSOS95del) control strain (Table III). 824(pCAS) also has low overall solvent formation and higher acid formation with limited acid reassimilation compared to 824(pSOS95del). These results are consistent with the previous ctfB asRNA strain (Tummala S B, et al., 2003, Journal of Bacteriology 185(12):3644-3653; Tummala S B, et al., 2003, Journal of Bacteriology 185(6): 1923-1934; each herein incorporated by reference in its entirety).

To determine if low THL levels were limiting the conversion of acetyl-CoA to butyryl-CoA in the wild-type strain without AAD over expression, the thl gene including its endogenous promoter was amplified from genomic DNA and ligated into the pIMP1 shuttle vector to create plasmid pTHL. Following the transformation of this plasmid into the wild-type strain, pH controlled bioreactors were used to characterize the strain. The metabolism of the 824(pTHL) is characterized by initial levels of high acid production, typical in clostridial fermentations, but there is only very limited acid reassimilation (Table III). Along with the elevated levels of acid production, there is a dramatic decrease in the levels of solvents produced. Additionally, there is a sharp decrease in the cell density of the culture and a plateau of the glucose uptake just a few hours following the peak butyrate production. This indicates, for example, that the cells cannot reassimilate butyrate promptly and the solvent genes cannot be induced to respond to the butyrate production, which leads to growth inhibition. It is hypothesized that, for example, aad overexpression using p_{ptb} would promote early butanol production and a means for preventing the accumulation of inhibitory butyrate concentrations.

Overexpression of AAD using p_{ptb} was analyzed with (strain 824(pTHLAAD)) and without (strain 824(pPTBAAD)) thl overexpression, and the fermentation data from the two strains are summarized in Table III. As a result of AAD overexpression, ethanol levels in 824(pPTBAAD) increased to 76 mM, more than three times the wild-type production. Additionally butyrate was nearly completely re-assimilated by this strain, while the final butanol titer was 160 mM. Acetate production in 824(pPTBAAD) was also very high reaching final levels of 124 mM. With the addition of THL overexpression, 824(pTHLAAD) shows a significant shift in product formation compared to 824(pPTBAAD). Ethanol production is reduced from 76 mM in 824(pPTBAAD) to 28 mM in 824(pTHLAAD). Acetate formation in 824(pTHLAAD) is also reduced to nearly half the level of 824(pPTBAAD). Butanol is produced at similar levels in both strains while THL overexpression causes a small increase in butyrate formation. Acetone levels were about 40% higher in 824(pTHLAAD) compared to 824(pPTBAAD).

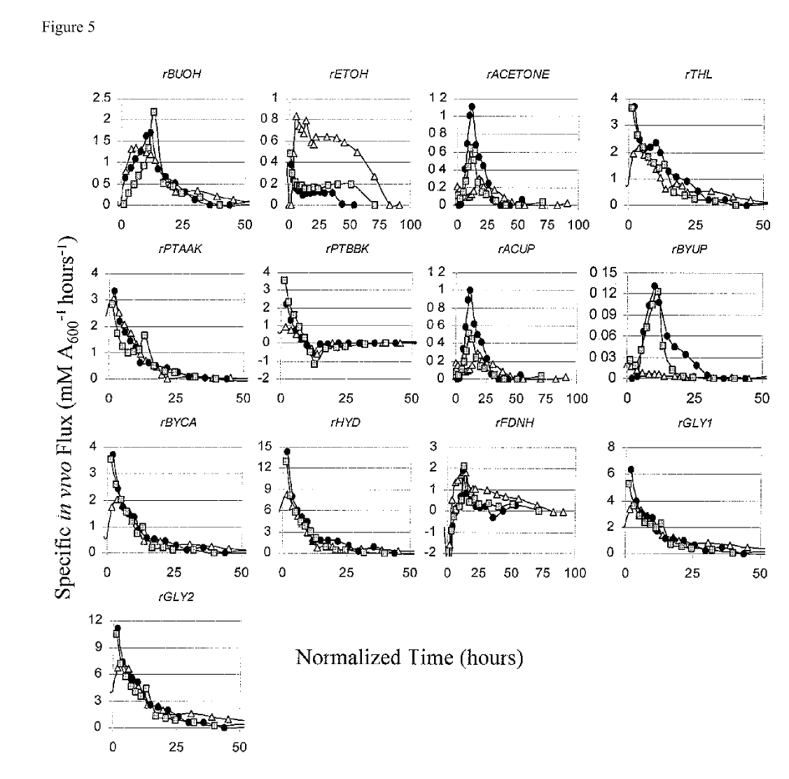

Comparing the profiles of the different fluxes (FIG. 5) provides additional insight into the role of THL overexpression. Consistent with the THL overexpression, there is an increase in the thiolase flux in 824(pTHLAAD) from about 5 hours to 30 hours (notice that the time scale in the flux analysis is in normalized hours) compared to 824(pPTBAAD). The higher butanol and BYCA fluxes early (in normalized hours) in the fermentation show that butanol is produced earlier in 824(pTHLAAD) than in 824(pPTBAAD) apparently because THL overexpression can enhance the butyryl-CoA rate of formation. The ethanol formation flux is similar between the two strains until about 25 hours into the fermentation when the flux is sharply reduced to zero at 40 hours in 824(pTHLAAD), while the ethanol formation flux is sustained at a high level in 824(pPTBAAD) after 50 hours. The HYD and FDNH fluxes are not affected by THL overexpression. Comparing the acid formation fluxes there appears to be little difference, but the acid uptake fluxes are significantly increased in 824(pTHLAAD) compared with 824(pPTBAAD): the acetate uptake flux is nearly twice as high in 824(pTHLAAD) and is sustained longer than in 824(pPTBAAD), while the butyrate uptake flux has a similar magnitude, but is sustained longer in 824(pTHLAAD). The acetone formation flux follows a similar pattern as the acetate uptake flux showing that acetone formation is mostly due to acetate uptake. Significantly, except for the first few hours, the BYCA flux is identical between the two strains. These flux analysis data then show that THL overexpression enhances acetoacetyl-CoA formation which enhances acetone formation and acetate uptake. Except for very early in the fermentation (in normalized hours), the lack of a major impact on the BYCA flux suggests that that flux is limited by one of the HBD, CRO or BCD enzymes (FIG. 1).

thl overexpression achieved the goal of reducing the acetylCoA pool and thus reduce the formation of ethanol and acetate. Indeed, in 824(pPTBAAD) the ratio of the concentrations of the two-carbon products (ethanol and acetate) to the four-carbon products (butanol and butyrate) was 0.81. When THL was overexpressed with AAD in 824(pTHLAAD), this ratio more than doubled to 1.79.

A comparison of the fermentation data (Table III) from strains 824(pPTBAAD) and 824(pCASAAD) illustrates the impact of the asRNA CoAT (FIG. 1) downregulation.

Combined Effect of THL and AAD Overexpression with CoAT Downregulation

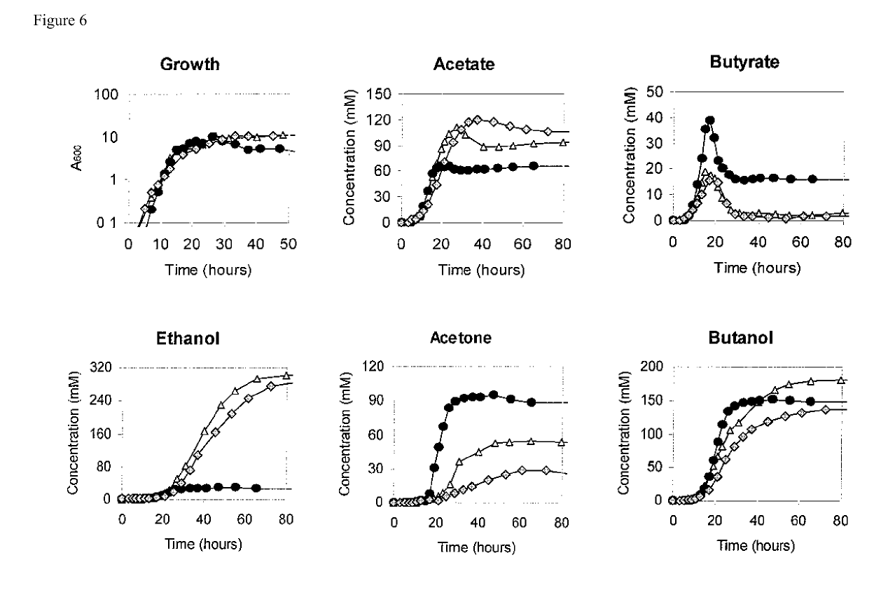

Plasmid pSS2 (Table I) was constructed to combine THL, AAD (from the p_{ptb}) overexpression, and CoAT downregulation by asRNA, but for the latter using the p_{ptb} instead of the pₜₕₗ used in the pCASAAD and pAADB1 plasmids. pH controlled fermentations of strain 824(pSS2) were once again used to characterize the strain in order to compare to the 824(pCASAAD) and 824(pTHLAAD) strains (FIG. 6). Strain 824(pSS2) grew a little slower than either 824(pCASAAD) or 824(pTHLAAD) and product formation was delayed even when normalized for differences in lag times; this is probably due to a general metabolic burden by the larger plasmid. Peak acetate production in 824(pSS2) was similar to 824(pCASAAD), but final acetate concentrations were higher. Butyrate formation was nearly identical in 824(pSS2) compared with 824(pCASAAD), which has lower peak and final butyrate levels than 824(pTHLAAD). Ethanol formation at 288 mM was very high in 824(pSS2), nearly as high as in 824(pCASAAD), which is much greater than ethanol production in 824(pTHLAAD). Acetone levels are much lower in the two strains harboring the ctfB asRNA, while butanol levels were fairly similar across all strains the with the lowest levels achieved in 824(pSS2). 824(pSS2) shows a more similar profile to 824(pCASAAD), which does not overexpress THL (but produces somewhat higher butanol and acetone levels) than 824(pTHLAAD), which does overexpress THL. These results indicate that the ctfB asRNA combined with AAD overexpression provide the dominant phenotype (high butanol and ethanol formation with suppressed acetone formation) that additional THL expression is unable to modulate in terms of enhancing butanol formation. It can also indicate that the p_{adc} driven asRNA CoAT downregulation has the desirable outcome, namely in producing a large suppression of acetone formation (which is fractionally larger than the suppression of either butanol or ethanol formation; compare the profiles of strain 824(pSS2) and 824(pCASAAD) in FIG. 6).

A comparison of strains 824(pSS2) and 824(pTHLAAD) demonstrates the impact of CoAT downregulation in the former is expected in that it reduces acetone formation, but unexpected in that it dramatically enhances ethanol and acetate formation apparently due to an increased acetyl-CoA pool. A similar conclusion is drawn following a comparison between strains 824(pPTBAAD) and 824(pCASAAD) (Table III): CoAT downregulation enhances dramatically ethanol formation but is accompanied by a lower final acetate production. pCASAAD has much higher ethanol and butanol formation fluxes, lower rTHL fluxes, dramatically lower acetate (rACUP) and butyrate (rBYUP) uptake fluxes, altered rFDNH, and altered acetate formation fluxes (higher early, lower later), all of which point to, for example, altered regulation around the acetyl-CoA node.

The pattern of aad expression was altered by replacing the endogenous promoter with that of ptb, which is responsible for butyrate formation. This caused both earlier and higher expression of aad and had marked effects on the fermentation products (FIGS. 2, 3 & 4). All the solvents (acetone, butanol, and ethanol) were produced at higher levels in 824(pCASAAD) with p_{ptb} driven aad expression than in 824(pAADB1), which uses the native aad promoter. The use of the ctfB asRNA kept acetone concentrations low, while ethanol concentrations reached the highest levels observed with this organism. The total solvent produced of 824(pCASAAD) is over 30 g/L with 13-14 g/L each of butanol and ethanol. Wild-type fermentations only produce about 20 g/L solvents, but acetone and butanol are the primary products. Additionally, butyrate is not totally reassimilated by the wild-type strain as it is in 824(pCASAAD). Final acid levels can be 15-25% of the total products in wild-type C. acetobutylicum fermentations, but in 824(pCASAAD) fermentations acids are only 5-10% of the total products. Other high solvent producing clostridia strains have been engineered that produced between 25-29 g/L total solvents in batch cultures, but again, the primary products are butanol and acetone (Harris L M, et al., 2000, Biotechnology and Bioengineering 67(1):1-11; Qureshi N, et al., 2001, J Ind Microbiol Biotechnol 27(5):287-91; Tomas C A, et al., 2003, Appl. Environ. Microb. 69(8):4951-4965; each herein incorporated by reference in its entirety). With the emergence of biofuels, strains producing ethanol may be preferred over those producing acetone as other significant products.

Metabolic flux analysis showed that the earlier expression of aad resulted in earlier formation of both butanol and ethanol. It also appears that butyryl-CoA depletion leads to the high ethanol yields. Ethanol production becomes significant as butanol production decreases due to reduced availability of butyryl-CoA. As the same enzyme (AAD; FIG. 1) catalyzes butanol and ethanol formation, genomic manipulations to directly decrease ethanol formation cannot be achieved by a simple metabolic engineering strategy. To further increase the butanol titers more of the acetyl-CoA (the precursor of ethanol) must be diverted to butyryl-CoA. Thiolase (THL) is the first enzyme in the conversion of acetyl-CoA to butyryl-CoA and its role in solvent production was investigated. THL overexpression combined with AAD overexpression lowered production of acetate and ethanol, while increasing acetone and butyrate levels. The ctfB asRNA used in the earlier studies was also redesigned to eliminate the use of the thl promoter to alleviate concerns of transcription factor titration effects. Although the combined THL and AAD overexpression does produce a substantial shift in the fermentation products, the combination of THL and AAD overexpression with CoAT asRNA downregulation does not significantly alter product formation compared to the strain without THL overexpression.

We claim:

1. A method for enhancing butanol production from a bacterial strain, comprising enhancing butyryl-CoA activity and diminishing acetyl-CoA activity in a bacterial strain.

2. The method of claim 1, wherein said bacterial strain is Clostridium acetobutylicum.

3. The method of claim 1, wherein said enhancing of butyryl-CoA activity is accomplished through overexpression of a bifunctional alcohol/aldehyde dehydrogenase gene.

4. The method of claim 3, wherein said bifunctional alcohol/aldehyde dehydrogenase gene is an alcohol/aldehyde dehydrogenase (aad) gene.

5. The method of claim 1, wherein said diminishing of acetyl-CoA activity is accomplished through targeting transcripts of enzymes in the acetone formation pathway with antisense RNA.

6. The method of claim 5, wherein said antisense RNA is ctfB antisense RNA.

7. The method of claim 3, wherein said overexpression of said bifunctional alcohol/aldehyde dehydrogenase gene is regulated via a promoter expressed during active cell growth.

8. The method of claim 5, wherein said antisense RNA is regulated via a promoter expressed during active cell growth.

9. The method of claim 7, wherein said promoter is selected from the group consisting of a phosphotranbutyrylase (ptb) promoter, a phosphotransacetylase (pta) promoter, and a thiolase (thl) promoter.

10. The method of claim 8, wherein said promoter is selected from the group consisting of a phosphotranbutyrylase (ptb) promoter, a phosphotransacetylase (pta) promoter, and a thiolase (thl) promoter.

11. The method of claim 1, wherein said diminishing of acetyl-CoA activity is accomplished through overexpression of a thiolase gene.

12. The method of claim 11, wherein said overexpression of thiolase is regulated via a promoter expressed during active cell growth.

13. The method of claim 12, wherein said promoter is selected from the group consisting of a phosphotranbutyrylase (ptb) promoter, a phosphotransacetylase (pta) promoter, and a thiolase (thl) promoter.

14. A method for increasing butanol production and reducing ethanol production in Clostridium acetobutylicum, comprising overexpression of the alcohol/aldehyde dehydrogenase gene and the thiolase gene in Clostridium acetobutylicum.

15. A method for increasing butanol and ethanol production in Clostridium acetobutylicum, comprising overexpression of the alcohol/aldehyde dehydrogenase gene and through inhibition of acetyl-CoA activity with ctfB antisense RNA in Clostridium acetobutylicum.


SEQUENCE LISTING (14 sequences)

SEQ ID NO: 1 - DNA, 22 bp, Artificial Sequence
  Synthetic
ttagaaagaagtgtatatttat

SEQ ID NO: 2 - DNA, 18 bp, Artificial Sequence
  Synthetic
aaacgacggccagtgaat

SEQ ID NO: 3 - DNA, 24 bp, Artificial Sequence
  Synthetic
ccatatgtcgacggaaaggcttca

SEQ ID NO: 4 - DNA, 24 bp, Artificial Sequence
  Synthetic
acgcctagtactgaattcgcctca

SEQ ID NO: 5 - DNA, 97 bp, Artificial Sequence
  Synthetic
tcgactaaaaatttacttaaaaaaacatatgtgttataatgaaatataaataaataggactagaggcgatttataatgtgaagataaagtatgttag

SEQ ID NO: 6 - DNA, 99 bp, Artificial Sequence
  Synthetic
aattctaacatactttatcttcacattataaatcgcctctagtcctatttatttatatttacattataacacatattgtttttttaagtaaatttttag

SEQ ID NO: 7 - DNA, 100 bp, Artificial Sequence
  Synthetic
aattcttaattctcttgcaactcttttggctattatttctttcgctaggtttttatcattaatcattttatgcaggctccttaaaagtaattacattaca

SEQ ID NO: 8 - DNA, 98 bp, Artificial Sequence
  Synthetic
tatgatatgtaattacttttaaggagcctgcataaaatgattaatgataaaaacctagcgaaagaaataatagccaaaagagttgcaagagaattaag

SEQ ID NO: 9 - DNA, 26 bp, Artificial Sequence
  Synthetic
tcgactaaaaatttacttaaaaaaac

SEQ ID NO: 10 - DNA, 24 bp, Artificial Sequence
  Synthetic
tatgatatgtaattacttttaagg

SEQ ID NO: 11 - DNA, 20 bp, Artificial Sequence
  Synthetic
agaaaatggctcacgcttca

SEQ ID NO: 12 - DNA, 24 bp, Artificial Sequence
  Synthetic
gcaatgccaactaggaatattgtg

SEQ ID NO: 13 - DNA, 22 bp, Artificial Sequence
  Synthetic
ttctccactgtggcgtagagtt

SEQ ID NO: 14 - DNA, 27 bp, Artificial Sequence
  Synthetic
tctctaagatcccaatctatccaattt